(19) United States
(12) Patent Application Publication
(10) Pub. No.: US 2008/0071260 A1
(43) Pub. Date: Mar. 20, 2008

(54) Electrosurgical generator and method using a high permeability, high resistivity transformer

(76) Inventor: Ronald B. Shores, Greenwood Village, CO (US)

Correspondence Address: JOHN R LEY, LLC, 5299 DTC BLVD, SUITE 610, GREENWOOD VILLAGE, CO, 80111, US

(21) Appl. No.: 11/521,710

(22) Filed: Sep. 15, 2006

(51) Int. Cl. A61B 18/18

(52) U.S. Cl. 606/34

(57) ABSTRACT

A transformer which conducts or responds to a high voltage, high frequency electrosurgical output waveform has a core with a permeability in the range of 500-2000 and a resistivity in the range of 90,000-1,000,000 ohm centimeters and insulation on the secondary high voltage winding of at least 800 VAC per 0.001 inch thickness. The permeability and resistivity of the core enhance energy conversion, reduce parasitic capacitance to enhance the high frequency spectral energy content of the electrosurgical output waveform while simultaneously reducing leakage current, reducing the size of the transformer, enhancing manufacturing reproducibility and enhancing the ability to pass a high voltage safety test.

This invention relates to electrosurgery, and more particularly to a new and improved electrosurgical generator and method for enhancing a high frequency, high voltage electrosurgical output waveform by using a high permeability, high resistivity transformer which conducts the electrosurgical output waveform, to obtain an increased bandwidth or spectral content of high-frequency energy in the electrosurgical output waveform, decreased leakage current, increased energy conversion efficiency, decreased sense signal distortion, smaller size and enhanced manufacturing repeatability, among other significant improvements.

### BACKGROUND OF THE INVENTION

Electrosurgery involves the application of a relatively high voltage and high frequency electrosurgical output waveform to living tissue during a surgical procedure. Depending upon its spectral energy content and other characteristics, the electrosurgical output waveform will cut tissue, stop or coagulate bleeding from the tissue, or will simultaneously cut and coagulate the tissue. The high frequency is typically within the 400 to 600 kHz range, because an electrical signal having this frequency does not stimulate the nervous system. The open circuit output voltage of the electrosurgical output waveform is typically in the range of 2,000 to 10,000 AC volts, peak to peak. The power of applied energy can vary from a few watts for coagulating delicate tissue to approximately 300 watts for cutting substantive tissue.

An electrosurgical generator creates the electrosurgical output waveform from conventional 110 or 220 volts AC commercial power. The electrosurgical generator converts low frequency, low voltage commercial power into the high frequency high voltage electrosurgical output waveform. The electrosurgical output waveform is applied from an active electrode manipulated by the surgeon at the surgical site. The active electrode may be part of a pencil-like handpiece, or a minimally invasive instrument manipulated through an endoscope or laparoscope. When monopolar electrosurgery is performed, a return electrode is electrically connected to the patient to create a return path through the tissue from the surgical site to the electrosurgical generator. The return electrode is a separate, relatively large pad attached to the skin of the patient at a location remote from the surgical site. When bipolar electrosurgery is performed, the return electrode is similar in size to the active electrode and both electrodes are part of a forceps-like device which squeezes the tissue between the active and return electrodes while the electrosurgical output waveform is conducted between the electrodes and through the squeezed tissue. Because of safety considerations, the electrosurgical output waveform is referenced to the patient and is isolated from the electrical ground-referenced components of the electrosurgical generator.

The electrosurgical generator uses a power output transformer to convert a low voltage, high frequency signal, applied to a primary winding of the power output transformer, into the high voltage, high frequency electrosurgical output waveform. The high voltage, high frequency electrosurgical output waveform is delivered from the secondary winding of the power output transformer. The characteristics of the power output transformer significantly influence the characteristics of the electrosurgical output waveform. The frequency-responsive impedance characteristics of the power output transformer determine the bandwidth or energy frequency spectrum of the electrosurgical output waveform. A typical power output transformer attenuates the high frequency spectral energy content of the electrosurgical output waveform. An attenuated energy frequency spectrum may negatively influence the ability of the electrosurgical output waveform to achieve the desired electrosurgical effect, particularly during coagulation. The power transfer characteristics of the power output transformer determine the energy conversion efficiency. Energy conversion efficiency is important in delivering adequate electrosurgical power to the tissue to respond to a wide variety of different and almost instantaneously-changing tissue impedances incurred during an electrosurgical procedure. The characteristics of the power output transformer also significantly influence the ability of the electrosurgical generator to operate safely and to comply with safety test regulations required by many governmental and quasi-governmental organizations.

In addition to the power output transformer, a typical electrosurgical generator uses other sense, signaling and isolation transformers which conduct and respond to the electrosurgical output waveform. Voltage and current sense transformers are connected to sense the output voltage and output current characteristics of the electrosurgical output waveform and transform the voltage and current of the isolated, patient-referenced electrosurgical output waveform to levels which are compatible with the low voltage, ground-referenced control components of the electrosurgical generator. Return electrode quality contact monitoring devices use one or two transformers to introduce or superimpose a monitoring signal and to sense the characteristics of the monitoring signal conducted through separate portions of the return electrode. Since the return electrode conducts the electrosurgical output waveform, the transformers which introduce and/or sense the monitoring signal must also conduct and respond to the electrosurgical output waveform while simultaneously shifting the monitoring signals from the ground-referenced components of the electrosurgical generator to the isolated patient-referenced electrosurgical output circuit which supplies the electrosurgical output waveform, and vice versa. In this sense, the transformers function as isolation transformers which separate the signals used in monitoring the return electrode contact from the electrosurgical output waveform. Mode selection transformers are also employed to sense mode selection signals from a switch on the handpiece which supports the active electrode. The mode selection switch conducts the electrosurgical output waveform to the mode selection transformers which transform the patient-referenced high voltage electrosurgical output waveform to the ground-referenced control components of the electrosurgical generator.

Because the power output transformer and the sense, signaling and isolation transformers all conduct and respond to the high frequency electrosurgical output waveform, these transformers have the capability to degrade and distort the characteristics of the electrosurgical output waveform and sense signals obtained from the electrosurgical output waveform as a result of inherent parasitic capacitances between the ground referenced winding and the output patient referenced winding of the transformer. Furthermore, because each of these output power, sense and signaling transformers is referenced to ground as well as to the patient, each transformer has the capability to conduct undesirable leakage current. In the sense relevant to the present invention, leakage current is that amount of current represented by the difference between the amount of current initially generated for delivery as the electrosurgical output waveform and the amount of current returned from the patient return electrode. The amount of current returned to the electrosurgical generator is less than the amount of current delivered, because leakage current is conducted from the patient-referenced circuit to ground reference through various parasitic capacitances both inside and outside of the electrosurgical generator. Leakage current can be a significant safety concern, because the leakage current may flow through the surgeon or other surgical personnel or through the patient to a ground-referenced structure such as the surgical table. In these cases, inadvertent burns may occur. In those cases where the leakage current does not interact with the surgical personnel, leakage current itself diminishes the performance of the electrosurgical generator.

Because of the significant influences on the electrosurgical output waveform from the output power, sense, signaling and isolation transformers, the characteristics of those transformers should be selected to achieve the best performance possible under compromised conditions. For example, the typical power output transformer used for coagulation has a resin-impregnated, powdered iron core. Small particles of powdered iron are embedded in a resin polymer to form the core of the transformer. The small gaps between the individual embedded particles greatly increase the inherent resistivity of the core, thereby supporting the high voltage of the electrosurgical output waveform without breakdown of the insulation on the high voltage conductors. The small gaps also store magnetic energy between the powdered iron particles for subsequent delivery. The small gaps also decrease the parasitic capacitance between the windings and the core. However, the distribution of ferromagnetic particles creates a relatively low permeability core, typically having a permeability value of about 85, which does not result in relatively efficient energy conversion. Permeability is the measure of how effectively flux flows through a material compared to the flow of that same flux through air. The permeability assigned to air is the value of 1. A higher permeability core will conduct more flux and therefore will generally be more efficient in energy conversion. Air core power output transformers are also sometimes used, because the high dielectric breakdown strength of air permits the air core to support very high voltages on the windings without breakdown of the insulation on those winding conductors. However, the low permeability of air makes the energy conversion efficiency very low. Consequently, the use of air core transformers is usually confined to relatively low power, low cost electrosurgical generators which have limited electrosurgical applicability. High permeability ferrite core material, which may exhibit a permeability of up to 10,000, provides a higher energy conversion efficiency, but high permeability ferrite core material is usually unsuitable for electrosurgical generators because it has a relatively low resistivity which makes it less capable of supporting the high voltage electrosurgical output waveform.

Increasing the number of windings on a transformer may compensate for the lower energy conversion efficiency of a low permeability core. However, increasing the number of windings increases the parasitic capacitance between individual coils or turns of the windings and between the coils and the core. At the typical high frequency of the electrosurgical output waveform, the relatively small coil-to-coil and coil-to-core parasitic capacitances become significant. At high frequencies, the parasitic capacitances remove high frequency energy from the electrosurgical output waveform and degrade the bandwidth and energy spectral characteristics to the point where electrosurgical performance may be adversely influenced, particularly in coagulation, or sensed signals may be compromised due to the distortion resulting from such capacitances. These parasitic capacitances also create a low impedance path to the ground reference and are thus responsible for a significant portion of the undesirable leakage current.

Increasing the number of coils or the thickness of the insulation on the winding conductors also increases the size of the transformer and complicates the ability to manufacture each transformer with repeatable characteristics. The increased insulation thickness physically spaces adjacent coils further from one another. As a result, the secondary winding consumes more space and typically requires a larger core. Because the physical placement of the windings on the core has a significant influence on the performance characteristics of the transformer, the larger size of the core and greater number of windings introduce manufacturing differences which change the characteristics of one transformer compared to another. A transformer core of increased size consumes more energy, because there are more molecular and crystalline components in the larger core to orient with the flux. The increased thickness of the electrical insulation spaces the coils at a greater distance from the core, which may allow some flux within the core to leak or escape without interacting with the windings, thereby diminishing energy conversion efficiency.

All of these various competing considerations lead to compromises when constructing any transformer, but the compromises are particularly significant with respect to electrosurgical transformers which conduct the high voltage, high frequency electrosurgical output waveform.

### SUMMARY OF THE INVENTION

The present invention improves transformers which conduct and respond to a high frequency, high voltage electrosurgical output waveform from an electrosurgical generator. The transformer of the present invention uses a core which exhibits both relatively high permeability and relatively high resistivity characteristics. The higher permeability of the core is greater than that of a resin-impregnated, powdered iron power output transformer typically used in electrosurgical generators. The increased permeability achieves greater energy conversion efficiency, which allows the number of windings to be reduced. While the higher resistivity of the core is not as great as that of a resin-impregnated, powdered iron power output transformer, the resistivity is sufficiently high to support a much higher voltage on the secondary winding without increasing the thickness of the insulation on the secondary winding conductor. Consequently, the secondary winding is more immune from arcing and discharge breakdown. The reduced number of windings reduces the parasitic coil-to-coil and coil-to-core capacitances. Reducing the parasitic capacitance avoids significantly distorting and degrading the bandwidth and high frequency spectral energy content of the electrosurgical output waveform. Leakage current is reduced because the diminished parasitic capacitance diverts less energy from the electrosurgical output waveform. The higher resistivity of the core also reduces the effects of parasitic coil-to-core and coil-to-coil impedances, thereby significantly increasing the core impedance to reduce leakage current. Moreover, the higher permeability of the core and the lesser number of windings with smaller insulation thicknesses, reduce the size of the transformer and enhances its manufacturing repeatability, because fewer numbers of components must be assembled in the same relative positions.

One aspect of the invention involves an electrosurgical generator which delivers a high frequency, high voltage electrosurgical output waveform for use in an electrosurgical procedure performed on a patient. The electrosurgical generator includes a transformer connected to respond to the electrosurgical output waveform. The transformer comprises a core around which primary and secondary windings are wound. The material forming the core has a permeability in the range of 500-2000 and a resistivity in the range of 90,000-1,000,000 ohm centimeters. Preferably, the permeability is in the range of approximately 800-2000 and the resistivity is in the range of 100,000-1,000,000 ohm centimeters.

Another aspect of the invention relates to the electrical insulation covering the secondary winding electrical conductor. The insulation has multiple layers of substantially uniform thickness. Preferably, the dielectric strength of each of the layers, and the dielectric strength of all layers of the insulation as a whole, is at least 800 VAC per 0.001 inch thickness (measured for insulation thicknesses under 0.010 inch thickness). Preferably the total thickness of the insulation is approximately 0.006 inch and each layer has a thickness of approximately 0.002 inch. The insulation of the secondary winding is preferably a fluoropolymer.

The transformer of the present invention may be the power output transformer of the electrosurgical generator, a sensing transformer which senses the voltage or current of the electrosurgical output waveform, a signaling or sensing transformer which supplies or senses a monitoring signal conducted by a return electrode to monitor contact of the return electrode with the patient, or an interrogation transformer which senses a signal from the electrosurgical output waveform supplied by a mode selection switch on a handpiece, among others.

Another aspect of the invention involves a method of increasing the high frequency energy content of a high frequency, high voltage electrosurgical output waveform delivered from an electrosurgical generator to a patient-referenced circuit, while simultaneously reducing leakage current from the electrosurgical output waveform and enhancing the resistance to arcing and glow discharge of the high voltage electrosurgical output waveform. The method involves connecting a secondary winding of a transformer to conduct the electrosurgical output waveform, and using material for a core of the transformer which has a permeability in the range of 500-2000 and a resistivity in the range of 90,000-1,000,000 ohm centimeters. The method also preferably includes insulating the electrical conductor which forms the secondary winding with electrical insulation having a dielectric strength of 800-2000 VAC per 0.001 inch of thickness of insulation (measured for insulation thicknesses under 0.010 inch thickness). In the manner described above, increasing the resistivity of the core and the dielectric strength of the insulation on the secondary winding electrical conductor reduces the amount of the electrical field which must be borne by the insulation and the adjacent air, thereby enhancing the high voltage withstanding capabilities of the transformer.

The invention also involves a method of increasing resistance to arcing and glow discharge through electrical insulation surrounding a secondary winding electrical conductor of a power output transformer of an electrosurgical generator. The electrical conductor which forms the secondary winding is insulated with multiple layers of electrical insulation. Each layer has a dielectric strength of 800-2000 VAC per 0.001 inch of thickness, measured for insulation thickness under 0.010 inches. Insulation strength ratings are based on the thickness of the test specimen. A core material of the transformer has a permeability in the range of 500-2000 and a resistivity in the range of 90,000-1,000,000 ohm centimeters.

A more complete appreciation of the present invention and its scope may be obtained from the accompanying drawings, which are briefly summarized below, from the following detailed descriptions of presently preferred embodiments of the invention, and from the appended claims.

### BRIEF DESCRIPTION OF THE DRAWINGS

FIG. 1 is a block and schematic diagram of an electrosurgical generator which utilizes a number of a high permeability, high resistivity transformers in accordance with the present invention.

FIG. 2 is a perspective view of an exemplary high permeability, high resistivity transformer which incorporates the present invention and which represents the transformers of the electrosurgical generator shown in FIG. 1.

FIG. 3 is a transverse cross-sectional view of the transformer shown in FIG. 2, taken substantially in the plane of line 3-3 in FIG. 2.

FIG. 4 is an enlarged cross-sectional view of an electrical conductor which forms a high-voltage winding on the transformer shown in FIGS. 2 and 3.

FIG. 5 is a cross-sectional view of an exemplary prior art transformer with a power ferrite core having relatively high permeability and relatively low resistivity, in which the core has a geometric configuration similar to that of the transformer shown in FIG. 3.

FIG. 6 is a perspective view of another type of an exemplary prior art transformer with a plastic- or resin-encapsulated ferrite particle or powder core having relatively low permeability and relatively high resistivity, in which the core has a geometric configuration of a toroid.

FIG. 7 is a cross-sectional view of the prior art transformer shown in FIG. 6, taken substantially in the cylindrically curved plane of line 7-7.

FIG. 8 is an enlarged view of a portion of the core and the secondary winding of the transformer shown in FIG. 3, taken substantially in the area bounded by line 8-8 in FIG. 3.

FIG. 9 is an enlarged view of a portion of the core and the secondary winding of the prior art transformer shown in FIG. 5, taken substantially in the area bounded by line 9-9 in FIG. 5.

FIG. 10 is an enlarged view of a portion of the core and the secondary winding of the prior art transformer shown in FIGS. 6 and 7, taken substantially in the area bounded by line 10-10 in FIG. 7.

FIG. 11 is a simplified impedance divider circuit diagram illustrating electrical impedance effects of the electrical insulation of the secondary winding and the impedance of the windings and the core shown in FIG. 8.

FIG. 12 is a simplified impedance divider circuit diagram illustrating electrical impedance effects of the electrical insulation of the secondary winding and the impedance of the windings and the core shown in FIG. 9.

FIG. 13 is a simplified impedance divider circuit diagram illustrating electrical impedance effects of the electrical insulation of the secondary winding and the impedance of the windings and the core shown in FIG. 10.

FIG. 14 is an exploded perspective view of an exemplary printed circuit board transformer which constitutes another example of the high permeability, high resistivity transformer of the present invention.

### DETAILED DESCRIPTION

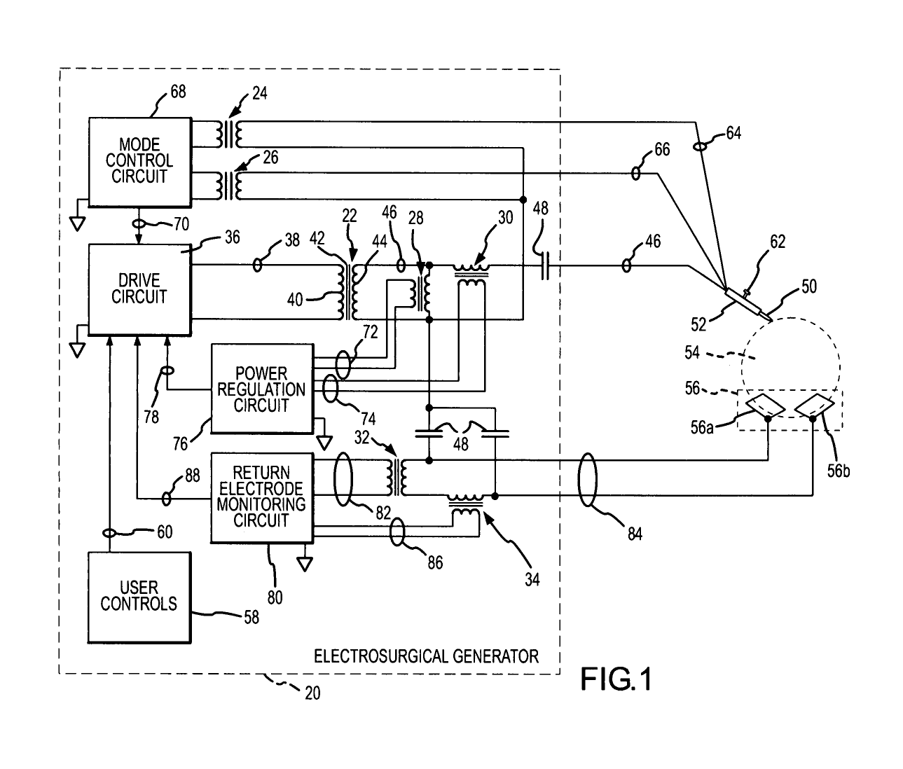

An electrosurgical generator 20, which incorporates one or more high permeability, high resistivity transformers in accordance with the present invention, is shown in FIG. 1. The high permeability, high resistivity transformers may be used as a power output transformer 22, a cut mode signal sense transformer 24, a coagulation mode signal sense transformer 26, an output voltage sense transformer 28, an output current sense transformer 30, a return electrode monitoring signal supply transformer 32, a return electrode monitoring signal sense transformer 34, and/or others which are not shown. The advantageous characteristics of the high permeability, high resistivity transformers 22, 24, 26, 28, 30, 32 and 34 create a significantly improved electrosurgical transformer and electrosurgical generator 20, for the reasons explained below.

The electrosurgical generator 20 includes a drive circuit 36 which supplies a primary drive signal 38 to a primary winding 40 of the power output transformer 22. The primary drive signal 38 causes current to flow in the primary winding 40, and that current causes magnetic flux in a core 42 of the transformer 22. The flux in the core 42 interacts with a secondary winding 44 of the transformer 22 to induce a signal 46 in the secondary winding 44. The signal 46 from the secondary winding 44 becomes an electrosurgical output waveform from the electrosurgical generator 20.

The electrosurgical output waveform 46 is conducted through an isolation capacitor 48 to an active electrode 50. The active electrode 50 is supported by a pencil-like handpiece 52, or the active electrode 50 is attached to a minimally invasive instrument (not shown), which the surgeon manipulates at a surgical site on a patient 54. The characteristics of the electrosurgical output waveform 46 cause it to cut tissue, to coagulate blood flow from the tissue, or to achieve a blend of simultaneous cutting and coagulation.

In monopolar electrosurgery, a return electrode 56 is connected to the patient 54 to collect current from the patient 54 and return the current through the isolation capacitors 48 to the electrosurgical generator 20, thereby completing a circuit through the tissue of the patient. In bipolar electrosurgery, the active and return electrodes are part of a forceps-like handpiece that contacts and squeezes tissue. The electrosurgical output waveform 46 is conducted through the tissue between the active and return electrodes of the forceps-like handpiece. In both monopolar and bipolar electrosurgery, the active and return electrodes 50 and 56 and the patient 54 are part of a patient-referenced electrical circuit that is isolated by the isolation capacitors 48 from the ground-referenced functional components of the electrosurgical generator 20. The electrosurgical output waveform 46 readily passes through the isolation capacitors 50 due to its high frequency characteristics.

The characteristics and energy content of the electrosurgical output waveform 46 are primarily established by the characteristics of the primary drive signal 38 and the energy conversion characteristics of the power output transformer 22. The characteristics of the primary drive signal 38 are established by the drive circuit 36 in response to power and mode selection signals 60 supplied by user controls 58. The user controls 58 are manipulated by the surgeon to select the desired electrosurgical mode of operation (cut, coagulate, or blended cut and coagulation) and the amount of power to be delivered in the selected electrosurgical mode. The selected mode of electrosurgical operation and the selected level of electrical power to be delivered cause the drive circuit 36 to deliver the drive signal 38 which obtains those selected characteristics.

In addition to the mode selection available from the user controls 58, the handpiece 56 may include a switch 62 that allows the surgeon to select between cutting and coagulation modes of operation. When the surgeon operates the switch 62 to select a cut mode of operation, a cut mode signal 64 is supplied from the handpiece 56 to the cut mode signal sense transformer 24. When the surgeon operates the switch 62 to select a coagulation mode of operation, a coagulation mode signal 66 is supplied from the handpiece 56 to the coagulation mode signal sense transformer 26. The mode signals 64 and 66 have the same high frequency and a comparable high voltage of the electrosurgical output waveform 46, because the switch 62 conducts part of the electrosurgical output waveform 46 to the transformers 24 and 26 as each mode signal 64 and 66. The sense transformers 24 and 26 respond to the mode signals 64 and 66, respectively, while isolating the mode signals from the electrosurgical output waveform and supply corresponding signals to a mode control circuit 68. The mode control circuit 68 supplies a mode control signal 70 to the drive circuit 36, causing the drive circuit 36 to deliver the drive signal 38 which has power characteristics selected by the user controls 58 and mode characteristics selected by the mode signals 64 and 66 from the switch 62.

A first winding of the output voltage sense transformer 28 is connected across the secondary winding 44 of the power output transformer 22. The other or second winding of the output voltage sense transformer 28 derives an output voltage sense signal 72 related to the magnitude of the voltage of the electrosurgical output waveform 46 conducted by the first winding. A first winding of the output current sense transformer 30 is connected in series with the secondary winding 44 of the power output transformer 22. The first winding of the transformer 30 conducts the current supplied by the electrosurgical generator 22 the active electrode 50. The other second winding of the output current sense transformer 30 derives an output current sense signal 74 related to the magnitude of the current conducted by the first winding. The output sense signals 72 and 74 are supplied to a power regulation circuit 76 of the electrosurgical generator 20.

The power regulation circuit 76 calculates the amount of power contained in the electrosurgical output waveform 46 based on the output voltage and current sense signals 72 and 74, and supplies a power feedback control signal 78 to the drive circuit 36. The drive circuit 36 responds to the feedback control signals 78 by adjusting the characteristics of the drive signal 38 to regulate the output power of the electrosurgical output waveform 46 in accordance with the level of power selected at the user controls 58. Although the power regulation circuit 76 is described as responding to the voltage and current of the electrosurgical output waveform 46, some types of electrosurgical generators regulate only with respect to the output voltage or with respect to the output current, or with respect to output voltage over a certain range of the load which the electrosurgical generator experiences and with respect to output current over a different range of the load.

Many electrosurgical generators incorporate a return electrode monitoring capability to evaluate the contact area or contact quality of the return electrode 56 with the patient 54. The return electrode monitoring capability is intended to avoid inadvertent burns to the patient 54 as a result of the return electrode 56 accidentally separating from the patient 54 by a significant amount during a surgical procedure. If the return electrode 56 separates from the patient 54, a reduced surface area exists from which to conduct the current from the patient. If the surface area is reduced sufficiently, the current density may become so high at the areas of diminished contact to burn the patient 54 at the return electrode 56.

To monitor the quality of the contact of the return electrode 56, the return electrode 56 is divided into two separated portions 56a and 56b. Both portions 56a and 56b are attached to the patient 54 and to the secondary winding 44 of the power output transformer 22 through the isolation capacitors 48. Both portions 56a and 56b collect the electrosurgical current from the patient 54 and return the current to the electrosurgical generator 20. A return electrode monitoring circuit 80 delivers a supply signal 82 to one winding of the return electrode monitoring signal supply transformer 32. The monitoring signal supply transformer 32 converts the supply signal 82 into a monitoring supply signal 84 which is supplied to the return electrode portions 56a and 56b from the other winding of the transformer 32. The supply signal 82 and the monitoring supply signal 84 are of the same frequency, and that frequency is significantly different from the high frequency of the electrosurgical output waveform 46. Consequently, the monitoring supply signal 84 is distinguishable from the electrosurgical output waveform 46.

The monitoring supply signal 84 is conducted to the return electrode portion 56a, through the patient tissue between the electrode portions 56a and 56b, and back from the return electrode portion 56b. The monitoring signal supply transformer 32 shifts the signal 82 from the ground reference of the return electrode monitoring circuit 80 to the reference of the isolated patient-referenced circuit electrode portions 56a and 56b which conduct the electrosurgical output waveform 46.

The extent to which the electrode portions 56a and 56b make contact with the patient 54 is determined by sensing the conductivity of the patient monitoring signal 84. Greater conductivity, which indicates lower tissue resistance between the electrode portions 56a and 56b, represents adequate contact of the electrode portions 56a and 56b. Lesser conductivity, which indicates greater tissue resistance between the electrode portions 56a and 56b, represents a potentially inadequate contact of the electrode portions 56a and 56b.

The monitoring supply signal 84 flows through one winding of the return electrode signal sense transformer 34, and a related sensed conductivity signal 86 is supplied from the other winding of the sense transformer 34 to the return electrode monitoring circuit 80. The sensed conductivity signal 86 and the monitoring signal 84 are both used by the return electrode monitoring circuit 80 to determine when the tissue resistance or impedance through the patient 46 between the electrode portions 56a and 56b is acceptable and not acceptable. If the resistance or the impedance reaches an unacceptable level indicative of reduced contact area of the return electrode 56, the return electrode monitoring circuit 80 supplies a contact quality control signal 88 to the drive circuit 36. The drive circuit 36 responds to the contact quality control signal 88 by disabling or otherwise limiting the primary drive signal 38 to terminate or limit the power of the electrosurgical output waveform 46, thereby preventing unintentional patient burns.

Although a return electrode monitoring signal sense transformer 34 is shown in FIG. 1 separate from the supply signal transformer 32, many electrosurgical generators utilize only a single isolation transformer 32. In that circumstance, the sensed conductivity signal 86 results from monitoring the influence on the supply signal 82 which occurs as a result of the variable resistance load between the electrode portions 56a and 56b. In this manner, a single transformer performs the functionality of both transformers 32 and 34.

Because the transformers 22, 24, 26, 28, 30, 32 and 34 each respond to and conduct the electrosurgical output waveform 46, these transformers may adversely influence the electrosurgical output waveform 46. In accordance with the present invention, the high permeability, high resistivity characteristics of the cores of these transformers advantageously avoid or significantly limit losses in power of the electrosurgical output waveform 46, maintain greater bandwidth or spectral frequency content of the waveform 46, avoid excessive leakage currents, and reduce or substantially eliminate distortion in the sensed signals derived from sense, signaling and isolation transformers, among advantageous improvements. The transformers 22, 24, 26, 28, 30, 32 and 34 do not load the waveform to create excessive power losses. These transformers do not create large parasitic capacitances which bleed power from the waveform 46 at high frequencies. The substantially reduced parasitic impedance between the primary and secondary windings substantially reduces the leakage current which can risk injury to the patient and the surgical personnel. While some prior art electrosurgical generators include transformers which have successfully limited some of these potentially adverse effects, none of the presently-known electrosurgical generators do so by using the high permeability, high resistivity transformers of the present invention.

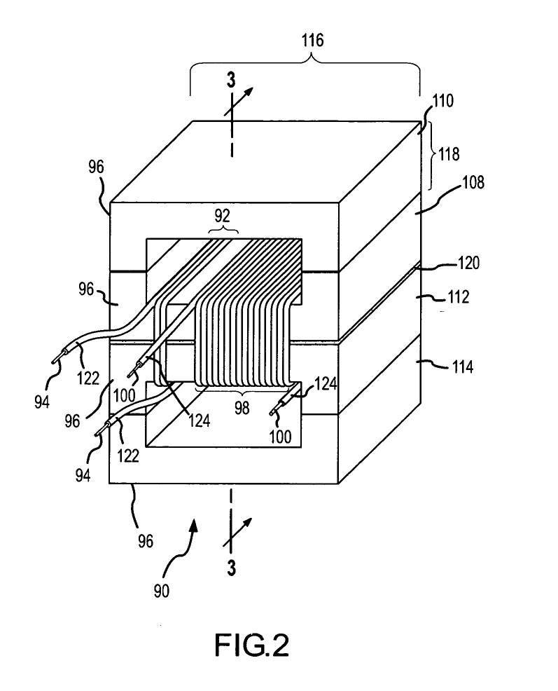

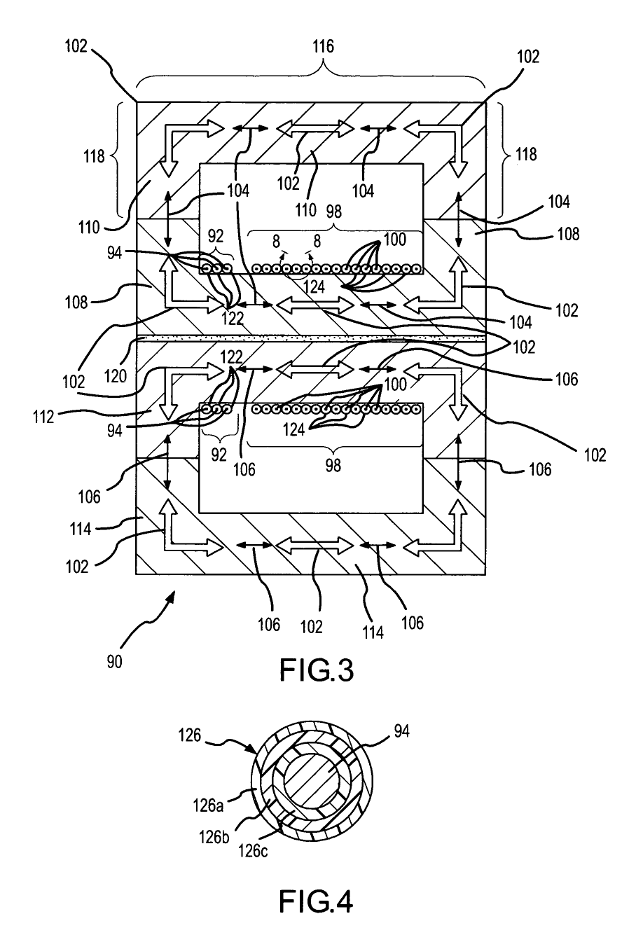

The high permeability, high resistivity characteristics of one and preferably all of the transformers 22, 24, 26, 28, 30, 32 and 34, and the improvements available in an electrosurgical generator which uses such high permeability, high resistivity transformers, are shown and discussed generically in connection with a single representative transformer 90 shown in FIGS. 2 and 3. The characteristics of the transformer 90 is intended to represent any of the transformers 22, 24, 26, 28, 30, 32 and 34.

The representative transformer 90 includes a primary winding 92 which is formed by an insulated primary electrical conductor 94 that has been wrapped or coiled around a core 96 in a predetermined number of windings, turns or coils. The transformer 90 also includes a secondary winding 98 which is formed by an insulated secondary electrical conductor 100 which has also been wrapped or coiled around the core 96 in a predetermined number of windings, turns or coils. To induce a secondary signal in the secondary winding 100, a primary signal is applied to the primary winding 92. The primary signal must have a changing current characteristic with respect to time in order to cause a corresponding change in magnetic flux 102 (FIG. 3) with respect to time within the core 96. The change in flux with respect to time in the core 96 induces a corresponding change in the secondary signal, to give the secondary signal a frequency characteristic the same as the frequency characteristic of the primary signal 38. The number of coils or turns of the primary winding 92 relative to the number of coils or turns of the secondary winding 98 establishes the voltage and current transformation characteristics of the transformer 90. The voltage of the secondary signal is ideally equal to the number of turns of the secondary winding divided by the number of turns of the primary winding multiplied by the voltage of the primary signal. The current of the secondary signal is ideally equal to the number of turns of the primary winding divided by the number of turns of the secondary winding multiplied by the current of the primary signal.

Energy from the primary signal is transformed into the energy of the secondary signal. Ideally, the electrical power of the primary signal (primary voltage multiplied by primary current) is transformed into an equal amount of electrical power in the secondary signal (secondary voltage multiplied by secondary current). In actual practice, some of the energy from the primary signal is consumed or dissipated without inducing the secondary signal. These losses prevent all of the energy from the primary signal from being converted into the energy of the secondary signal and thereby diminish the energy conversion efficiency of the transformer.

Energy conversion efficiency relates to the amount of magnetic flux which is generated by the primary signal. The flux generated by the primary winding which intercepts and interacts with the secondary winding is referred to as mutual flux, because that flux mutually interacts with both the primary and secondary windings. The amount of flux generated by the primary winding that escapes without intercepting and interacting with the secondary winding is known as leakage flux, because it is not available to induce the secondary signal. Leakage flux diminishes the energy conversion efficiency of the transformer.

Conducting flux in the transformer core involves orienting its molecular and crystalline constituent components. The energy to orient the molecular and crystalline components is supplied by the primary signal. The constituent components of the core are continually reoriented because of the changing flux in the core, causing the core to continually consume energy. This consumed energy is dissipated as heat, which also diminishes the energy conversion efficiency.

The core 96 of the transformer 90 is formed in a predetermined geometric configuration, preferably one which conducts the flux 102 in at least one closed flux flow path. The geometric configuration of the core 96 shown in FIGS. 2 and 3 creates two magnetic flux flow paths 104 and 106, each generally in the shape of a rectangle (FIG. 3). The core 96 is formed by assembling four identical U-shaped core pieces 108, 110, 112 and 114. Each core piece 108, 110, 112 and 114 includes a relatively long central portion 116 from which two relatively short leg end portions 118 extend perpendicularly in the same direction at the opposite ends of the central portion 116. The U-shaped core pieces 108 and 112 are connected with their central portions 116 in a back-to-back relationship with a layer of adhesive 120 holding the central portions 116 together. When connected in this manner, the leg end portions 118 of each core piece 108 and 112 extend in opposite directions from the leg end portions 118 of the other adhered-together core pieces 110 and of 114.

The primary winding 92 is formed by winding the primary conductor 94 in a predetermined number of coils around the two back-to-back adhered central portions 116 of the core pieces 108 and 112. The secondary winding 98 is formed by winding the secondary conductor 100 in a predetermined number of coils around the two back-to-back adhered central portions 116 at a position which is longitudinally spaced from the primary winding 92. A thin layer of insulating tape (not shown) may be wrapped around the central portions 116 of the pieces 108 and 112 before the primary and secondary conductors 94 and 100 are wrapped around the core pieces 108 and 112, to prevent the corners of the core pieces 108 and 112 from penetrating into the electrical insulation 122 and 124 which surrounds the primary and secondary conductors 94 and 100. A layer of electrical insulating tape (not shown) may also cover the exterior of the primary and secondary conductors 94 and 100 after the primary and secondary windings 92 and 98 have been formed.

After the primary and secondary windings 92 and 98 have been formed on the core pieces 108 and 112, the core pieces 110 and 114 are positioned to complete the two rectangularly-shaped flux flow paths 104 and 106 (FIG. 3). The leg end portions 118 of the core pieces 110 and 114 adjoin the complementary leg end portions 118 of the core pieces 108 and 112, thereby completing the flow path 104 for the flux 102 through the core pieces 108 and 110 and completing the flow path 106 for the flux 102 through the core pieces 112 and 114. The adjoining ends of the leg end portions 118 of the core pieces 108, 110 and 112, 114 may be connected with an adhesive or held together with exterior mechanical supports (not shown). To the extent that it may be desirable to modify the flux flow paths 104 and 106 through the core pieces 108, 110 and 112, 114, gaps may be formed between the contacting ends of the leg end portions 118. These gaps may momentarily store energy for subsequent release, for example. The current flow in the primary electrical conductor 94 induces the flux 102 in the flux flow paths 104 and 106 through the core pieces 108, 110 and 112, 114. The flux 102 in the flow paths 104 and 106 induces the secondary signal in the secondary winding 100.

While the specific geometric configuration of the core 96 may vary, and while it is preferable to establish at least one closed loop flow path 104 or 106 for the flux 102 within the core 96, the material from which the core 96 is formed (the core pieces 108, 110, 112 and 114) is an important aspect of the present invention. The core 96 and the core pieces 108, 110, 112 and 114 are formed from a magnetic material which simultaneously exhibits both a relatively high permeability and a relatively high resistivity. Such high permeability, high resistivity material is used principally for electromagnetic interference (EMI) suppression purposes. The high permeability, high resistivity core material is specifically formulated and is not a naturally-existing material. High permeability, high resistivity magnetic material is not known to have been used for transformers in electrosurgical generators which conduct or respond to the high voltage, high frequency electrosurgical output waveform.

In general, a higher permeability core in a transformer will conduct more flux compared to a lower permeability core. As noted above, permeability is the measure of how effectively flux flows through a material compared to the flow of that same flux through air. The permeability assigned to air is the value of 1. The permeability of conventional power conversion transformer ferrite core materials may approach 10,000. Because of its greater capability to conduct flux, a high permeability transformer core will usually allow less leakage flux, thus yielding greater energy conversion efficiency at the low frequencies of typical commercial power conversion. On the other hand, high permeability power ferrite core material is prone to higher internal energy losses which reduce energy conversion efficiency. Transformers with high permeability cores are typically used in circumstances where the frequency of the signal is relatively low (e.g. 50-60 Hz), where great quantities of electrical power must be converted and where the increased internal losses are tolerable. These circumstances are not generally applicable to an electrosurgical generator which must conduct or respond to the high frequency electrosurgical output waveform, which supplies relatively low power (compared to commercial power conversion), and which requires relatively high energy conversion efficiency due to the size and capability of the equipment.

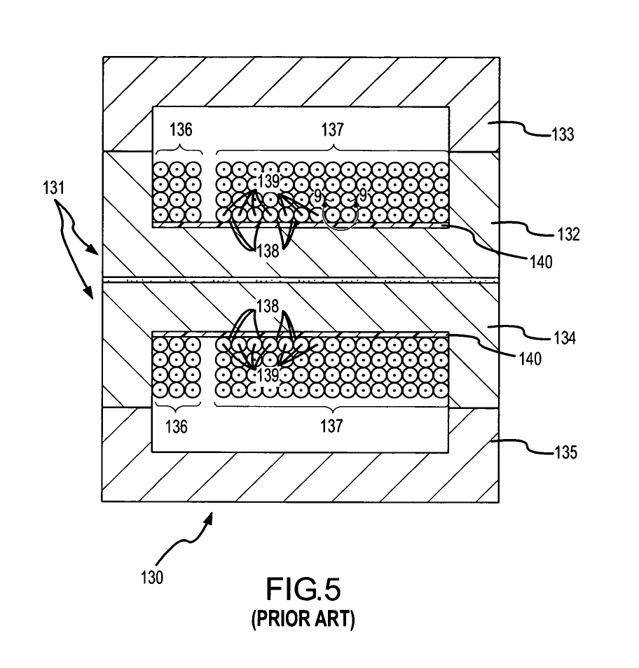

Preferably, the permeability of the core 96 and the core pieces 108, 110, 112 and 114, is at least 500. More preferably, the permeability is in the range of 800-2000. At the present time, a permeability of 2000 approximately defines the high end of the high permeability, high resistivity core material now available. The preferred permeability of the core 96 is therefore considerably higher than the typical permeability of about 85 for a resin-encapsulated powdered ferrite core transformer core (FIGS. 6 and 7) frequently used for power conversion in electrosurgical generators, but is less than the typical permeability of about 3000-10,000 for ferrite core material typically used for power conversion transformers but sometimes employed in electrosurgical generators (FIG. 5).

Another important characteristic of the core 96 is its high resistivity characteristic. The resistivity of the core refers to the capability of the core material to conduct current. Relatively low resistivity in a transformer core diminishes the level of voltage that the secondary winding can withstand without arcing or discharging through the insulation on the high voltage winding conductor. Relatively low resistivity of the core encourages arcing or discharging from the high voltage winding, since low core resistivity offers less resistance between the high voltage winding conductor and the ground reference. Even under circumstances where arcing through the secondary winding insulation does not occur immediately, a low resistivity core encourages a corona-type glow discharge through the secondary winding insulation to the core which, over time, will eventually deteriorate the insulation on the high voltage winding conductor to the point where arcing will begin. A high permeability power conversion ferrite core material typically has a resistivity within the range of 50 to 1000 ohm centimeters.

Preferably the core 96 has a resistivity of at least 90,000 ohm centimeters, and more preferably, has a resistivity in the range of 100,000-1,000,000 ohm centimeters. At the present time, 1,000,000 ohm centimeters approximately defines the highest known resistivity available for high permeability, high resistivity core material. The resistivity of the core 96 is not as high as that of a typical resin-encapsulated powdered iron core transformer used in electrosurgical generators (FIGS. 6 and 7) and is not nearly as high as the resistivity of an air core transformer used in some electrosurgical generators. However, the resistivity of the core 96 is considerably higher than the typical resistivity of power ferrite core material.

One important characteristic of the present invention relates to the relatively high permeability and relatively high resistivity of a core and a transformer of an electrosurgical generator. Core materials previously used in electrosurgical generators required a choice of either low permeability material which had high resistivity, as exemplified by a prior art transformer 130 shown in FIG. 5, or high permeability material which had low resistivity, as exemplified by a prior art resin-encapsulated powdered iron transformer 141 shown in FIGS. 6 and 7. The improvement resulting from the use of core material with both high permeability and high resistivity offers significant benefits when conducting and responding to the high-voltage, high frequency electrosurgical output waveform 46 of an electrosurgical generator 20 (FIG. 1).

Another desirable aspect of the present invention relates to the electrical insulation 124 which surrounds the high voltage winding conductor, typically the secondary winding conductor 100. Preferably the dielectric strength of the insulation on the secondary conductor 100 is in the range of 800-2000 VAC per 0.001 inch thickness for insulation thicknesses under 0.010 inch of thickness, due to insulation strength ratings being based on the thickness of the test specimen. Because of this relatively high dielectric strength per unit of thickness, the uniformity of the thickness of the electrical insulation 124 on the high-voltage winding can be reduced. For example, the substantially uniform thickness of the secondary winding insulation with the preferred range of dielectric strength may be in a range from approximately 0.006 inches thick to 0.015 inch thick. The typical high voltage winding insulation dielectric strength is approximately 400 VAC per 0.001 inch thickness in a typical prior art electrosurgical generator, resulting in a secondary winding insulation thickness of at least 0.045 inch.

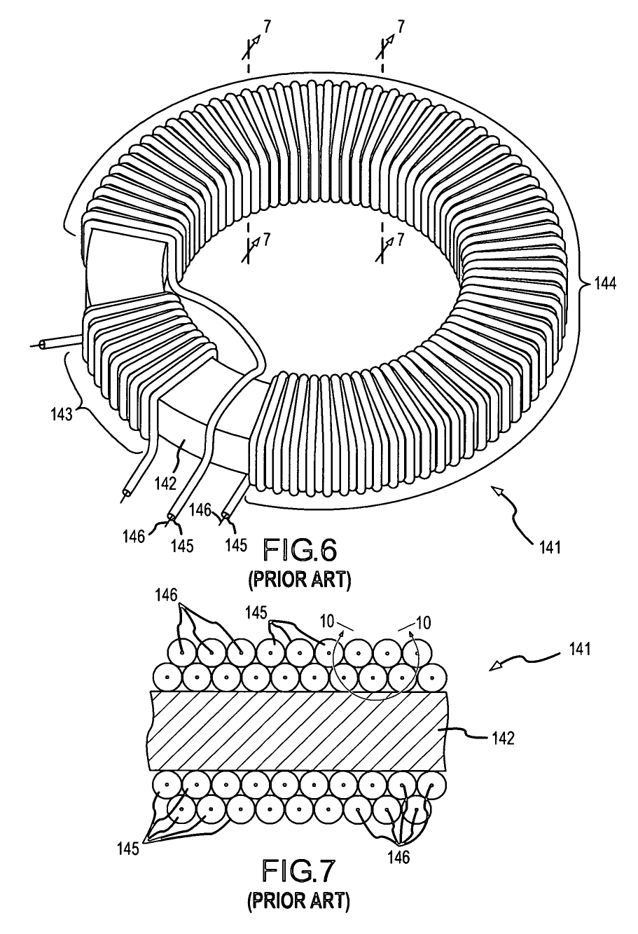

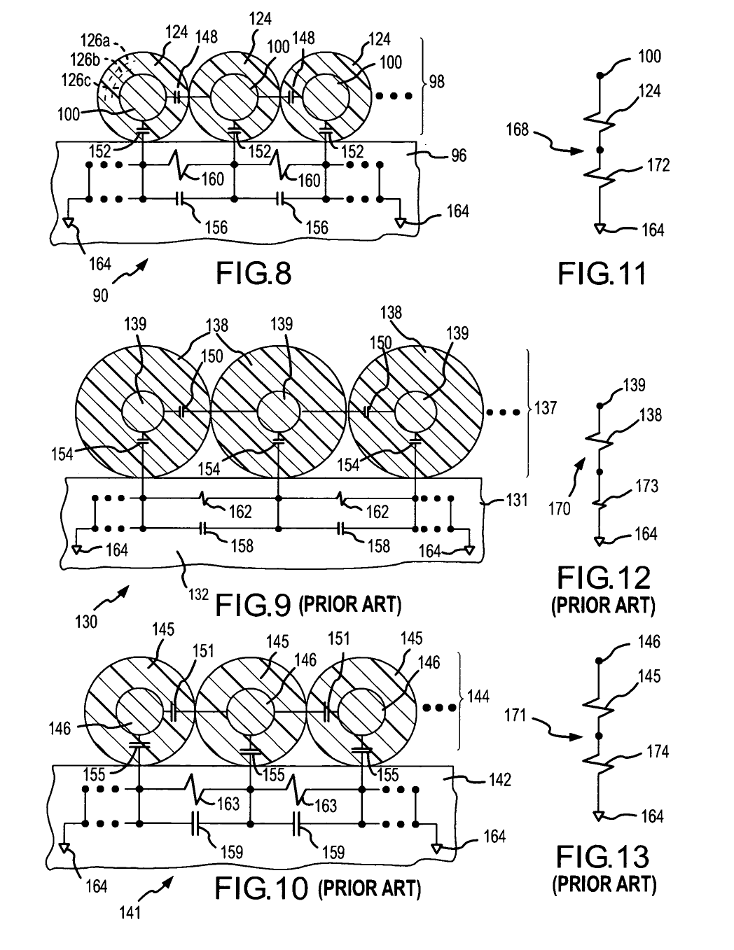

The insulation 124 around the high-voltage or secondary winding conductor 94 is preferably formed in multiple, contiguous layers 124a, 124b and 124c, as shown in FIGS. 4 and 8. Each such layer is substantially uniform in thickness and is approximately 0.002 inch thick. Forming the insulation 124 in multiple contiguous uniform-thickness layers enhances the dielectric strength and creates greater dielectric integrity within the entire amount of insulation 124. Each of the layers and the whole of the insulation 124 are preferably formed from Teflon® (PTFE) or Tefzel®, which are fluoropolymers manufactured by DuPont. Insulated high voltage conductors suitable for use as the high voltage or secondary winding conductor 100 are available from Rubadue Wire Co., of Greeley, Colo., USA.

The aspects of the transformer 90, with its high permeability, high resistivity core 96 and the thinner, high dielectric strength insulation 124, are better understood by comparison to a prior art power ferrite core transformer 130, shown in FIG. 5, and a prior art resin- or plastic-encapsulated ferrite particle core transformer 141, shown in FIGS. 6 and 7.

The prior art transformer 130 shown in FIG. 5 has a core 131 which is formed from high permeability, low resistivity power ferrite material. The core 131 comprises core pieces 132, 133, 134 and 135 which are assembled to create a rectangular geometric configuration. A primary winding 136 and a secondary winding 137 surround the back-to-back core pieces 132 and 134. The permeability of the power ferrite material of the core 131 is typically greater than the permeability of the core 96 of the transformer 90 of the present invention (FIG. 3), because the typical permeability of power ferrite material is greater than the typical permeability of the high permeability, high resistivity material which forms the core 96 (FIG. 3). On the other hand, the resistivity of the power ferrite material of the core 131 is substantially less than the resistivity of the high permeability, high resistivity material of the core 96 (FIG. 3).

The thickness of insulation 138 which surrounds a secondary electrical conductor 139 that forms the secondary winding 137 of the transformer 130 is substantial, because the insulation 138 must withstand a significant portion of the high voltage induced in the secondary winding electrical conductor 139, due to the relatively low resistivity of the core 131. The relatively low resistivity characteristic of the core 131 does not offer substantial assistance in inhibiting arcing of the relatively high voltage from the secondary conductor 139 to the core 131, thereby requiring the insulation 138 around the secondary conductor 139 to have substantial thickness to achieve enough dielectric strength to withstand arcing to the core 131.

The dielectric strength per unit of thickness of the insulation 138 surrounding the secondary conductor 139 is significantly less than that of the insulation 124 of the transformer 90 (FIG. 3). The lower dielectric strength per unit of thickness requires more thickness of the insulation 138 to achieve the same total dielectric strength as the insulation 124 (FIG. 3). A layer of insulating tape 140 may be wrapped around the core 131 to provide additional electrical insulation and dielectric strength to inhibit arcing from the secondary winding to the core 131.

The relative thicknesses of the insulation 124 and 138 around the secondary winding conductors 100 and 139, and the relative spacing between the secondary winding conductors 100 and 139 and from the secondary winding conductors 100 and 139 to the cores 96 and 131 in the transformers 90 and 130 are shown in FIGS. 8 and 9, respectively. The thicker insulation 138 spaces each of the coils of the secondary winding 137 further from each adjoining coil and further from the core 131, as shown in FIG. 9, compared to the closer spacing of the coils of the secondary winding 92 to each other and to the core 96, as shown in FIG. 8. The greater spacing of the secondary winding conductors 139 provides a greater opportunity for more flux to leak from the transformer 130 without interacting with the windings. The flux which leaks from the transformer 130 without interacting with the windings is not available to induce or create a signal in the winding and therefore diminishes the energy conversion efficiency of the transformer.

To overcome the effects of the leakage flux, the number of coils of the secondary winding conductor 139 is increased to attempt to create and intercept more of the flux, as shown in FIG. 5. Consequently, even though the permeability of the core material may be relatively high, an increased number of windings on the core 131 may nevertheless be required because the thicker insulation on the secondary winding conductor which gives rise to greater leakage flux.

The greater number of coils of the windings and the thicker insulation on the windings may cause the core 131 to be physically large in size to accept those windings, thus increasing the overall size of the transformer 130. A larger core 131 is more costly and will consume more energy in inherent core losses as a result of the flux flowing through a greater amount of core material. The increased number of coils of the primary and secondary windings 136 and 137 also increases the length of the conductors through which the signals are conducted, thereby diminishing the energy content of those signals due to the increased length of the power-consuming resistance path through which those signals are conducted.

The prior art transformer 141 shown in FIGS. 6 and 7 has a toroidal-shaped core 142 formed from encapsulating many powder-like particles of high permeability ferrite material in plastic material, such as epoxy or resin. The permeability of the core 142 is considerably less than the permeability of the core 96 (FIG. 3) or the core 131 (FIG. 5). The plastic material in which the individual particles of ferrite material are encapsulated as a very low permeability and high resistivity. The individual ferrite particles contribute to the permeability of the core 142, but the gaps between the individual ferrite particles do not establish a continuous flux flow path within the core 142. Therefore, the overall permeability of the core is relatively low. The spaces or gaps between the individual ferrite particles also prevent a continuous conductivity path through the core 142, thereby making the resistivity of the core 142 very high. Consequently, the overall permeability of the core is relatively low and the resistivity is relatively high. As a consequence of the relatively low permeability, a significant number of turns or coils of a primary winding 143 and a secondary winding 144 are required.

The thickness of insulation 145 which surrounds a secondary electrical conductor 146 of the secondary winding 144 will typically be moderate and less than the thickness of insulation 138 which surrounds the secondary winding conductor of the power ferrite transformer 130 (FIG. 5). However, the thickness of the insulation 145 will be greater than the thickness of the insulation 124 which surrounds the secondary winding conductor of the high permeability, high resistivity transformer 90 of the present invention (FIG. 3). The thickness of the insulation 145 is moderate because it is not required to withstand a great portion of the high voltage induced in the secondary electrical conductor 146, due to the relatively high resistivity of the core 142. The relatively high resistivity of the core 142, caused by the gaps between the ferrite particles, and the relatively high dielectric strength of the plastic material which encapsulates those ferrite particles, assists the insulation 145 in inhibiting arcing of the relatively high voltage from the secondary conductor 146.

Even though the thickness of the insulation 145 is moderate, the dielectric strength of the insulation 145 is less than the dielectric strength of the insulation 124 of the secondary winding 98 of the transformer 90 (FIG. 3). The lesser dielectric strength of the insulation 145 per unit of thickness also contributes to the greater total thickness of the insulation 124.

The relative thicknesses of the insulation 124 and 145 around the secondary winding conductors 100 and 146, and the relative spacing between the secondary winding conductors 100 and 146 and from the secondary winding conductors 100 and 146 to the core 96 and 142 in the transformers 90 and 130 are shown in FIGS. 8 and 10, respectively. The relatively lower permeability of the core 142 diminishes the flux carrying capability to the point where considerably more coils or turns of the windings 143 and 144 are required. The greater number of windings increases the parasitic capacitance between the windings, and the increased number of windings increases the parasitic capacitance of the windings as a whole with respect to the conductive metal particles embedded within the core 142. These parasitic capacitances create a significant degradation in the bandwidth or spectral energy content of the high frequency electrosurgical output waveform and on the sensed signals from the sense, signaling and isolation transformers. A reduced high frequency energy content of the electrosurgical output waveform diminishes some types of electrosurgical effects, such as coagulation, and also reduces the power conversion efficiency of the transformer itself.

The amount of plastic encapsulating the amount of ferrite particles necessary to create at least the low but usable level of permeability while simultaneously providing sufficient gaps between the ferrite particles to increase the resistivity of the core 142, and the number of coils or turns of the primary and secondary windings 143 and 144 necessary because of the reduced permeability, and the typical toroidal shape of the core 142 itself causes the transformer 141 to be physically large in size. The larger number of coils of the primary and secondary windings 143 and 144 also increases the length of the conductors through which the primary and secondary signals are conducted, thereby diminishing the energy content of those signals due to the increased length of the power-consuming resistance path through which those signals are conducted.

The beneficial electrical effects of the high permeability, high resistivity core 96 of the transformer 90 of the present invention (FIGS. 3 and 8) may be better understood when compared to the electrical effects of the high permeability, low resistivity core 131 of the prior art transformer 130 (FIGS. 5 and 9) and to the low permeability, high resistivity core 142 of the prior art transformer 141 (FIGS. 6, 7 and 10). In general, the relative thicknesses of the insulation 124, 138 and 145 previously described are shown in FIGS. 8-10, respectively.

Referring principally to FIGS. 8-10, and recognizing that the individual coils of the secondary winding electrical conductors 100, 139 and 146 of the secondary windings 98, 137 and 142 experience different voltages, it becomes apparent that coil-to-coil capacitances 148, 150 and 151 exist between the individual turns or coils of the secondary winding conductors 100, 139 and 146, respectively. The coil-to-coil capacitances 148, 150 and 151 are parasitic, meaning that energy from the slightly different signals between the individual coils of the secondary winding conductors 100, 139 and 146 must charge the coil-to-coil capacitances 148, 150 and 151, thereby diminishing the energy available to be delivered from the windings 98, 137 and 144. The amount of parasitic coil-to-coil capacitances depends on many factors, including the number of windings, the physical spacing of the windings, the thickness of the insulation 124, 138 and 145 surrounding the secondary winding conductors, and the dielectric strength of the insulation.

Coil-to-core parasitic capacitances 152, 154 and 155 also exist between the secondary winding conductors 100, 137 and 146 and the cores 96, 131 and 142, respectively. The coil-to-core parasitic capacitances 152, 154 and 155 are also parasitic and must also be charged with energy from the signals induced in the secondary winding conductors 100, 139 and 146. Charging these parasitic coil-to-core capacitances 152, 154 and 155 also has the effect of diminishing the energy available to be delivered from the windings 98, 137 and 144. The amount of parasitic coil-to-core capacitance also depends on many factors, including the number of windings adjacent to the core, the physical spacing of the windings adjacent to the core, the thickness of the insulation 124, 138 and 145 surrounding the secondary winding conductors 100, 139 and 146 adjacent to the core, the dielectric strength of the insulation, and the impedance characteristics of the core materials and 96, 131 and 142.

The materials forming the cores 96, 131 and 142 have inherent capacitance and resistivity. Since each of the coils of the secondary winding conductors 100, 139 and 146 carries a different voltage than its adjacent coil, the cores 96, 131 and 142 experience this different voltage at the different locations adjacent to the individual coils. The inherent capacitance and resistance of the cores 96, 131 and 142 is represented by distributed capacitance elements 156, 158 and 159 and by distributed resistance elements 160, 162 and 163.

The core capacitive elements 156, 158, 159 and the core resistance elements 160, 162, 163 are illustrated in FIGS. 8-10 as connected in series relative to reference potential 164. The reference potential 164 is located at remote points on the cores 96, 131 and 142 that are not encircled by the primary or secondary windings. The core capacitive elements 156, 158 and 159 and the core resistive elements 160, 162 and 163 are shown connected in series between points on the cores 96, 131 and 142 which are adjacent to each of the secondary winding conductors 130, 139 and 146 and between the reference potential 164. The series connection between points on the cores which are adjacent to each of the secondary winding coils represents the recognition that each of these core capacitive and resistive elements experiences a slightly different voltage since each of the secondary winding conductors 100, 139 and 146 experiences a slightly different voltage.

The magnitude of the core capacitive elements 156, 158 and 159 is usually relatively low in each case, and therefore does not significantly influence the entire core impedance represented by the parallel connection of the core capacitive elements 156, 158, 159 and the core resistive elements 160, 162, 163. However, the magnitude of the core capacitive elements 156, 158 and 159 varies in accordance with the frequency of the signal conducted through the windings. At the relatively high frequency of the electrosurgical output waveform, there is a slight to moderate reduction in the impedance of the cores as a result of the reduced high-frequency impedance from the core capacitive elements 156, 158 and 159. On the other hand, the magnitude of the core resistive elements 160, 162 and 163 has a considerably greater and more significant influence on the electrical characteristics of each of the transformers 90, 130 and 141. The resistance value of the inherent core resistance elements 160, 162 and 163 does not vary in relation to the frequency of the electrosurgical output waveform.

At the high frequency of the electrosurgical output waveform 46 (FIG. 1), the inherent impedance of the cores 96, 131 and 142 and the dielectric strength of the insulation 124, 138 and 145 on the secondary winding conductors 100, 139 and 146 must withstand the high voltage on the secondary windings 98, 137 and 146, respectively. This impedance must resist arcing and discharge from the secondary winding conductors 100, 139 and 146. These effects are illustrated by simplified two-element impedance divider circuits 168, 170 and 171, shown in FIGS. 11, 12 and 13. The impedance divider circuits 168, 170 and 171 correspond to certain electrical characteristics of the transformers 90 (FIG. 3), 130 (FIG. 5) and 141 (FIGS. 6 and 7) illustrated in FIGS. 8-10, respectively.

The two-element impedance divider circuits 168, 170 and 171, shown in FIGS. 11-13, illustrate the impedance effects of the insulation 124, 138 and 145 and relative to the combined distributed impedance effects of the parasitic and inherent capacitive and resistance elements of each transformer 90, 130 and 141. The combined impedance of these parasitic and inherent capacitive and resistance elements 148, 152, 156, 160, and 150, 154, 158, 162, and 151, 155, 159, 163 constitutes the impedance of elements 172, 173 and 174 in the divider circuits 168, 170 and 171, respectively. The magnitude of the impedance of the elements 172, 173 and 174 partially depends on the frequency of the signals conducted, since the impedance elements 172, 173 and 174 include the parasitic coil-to-coil capacitances 148, 150, 151, and the coil-to-core capacitances 152, 154, 155, as well as the inherent core capacitances 156, 158, 159, although as mentioned above, the inherent core capacitances 156, 158 and 159 generally have only a slight effect. The other component of the impedance of elements 172, 173 and 174 is the inherent core resistance 160, 162 and 163 which remains essentially constant independent of the frequency conducted by the transformer. The other impedance elements 124, 138, 145 of the divider circuits 168, 170 and 171 are the resistance value of the insulation 124, 138 and 145 surrounding the secondary winding conductors 100, 139 and 146, respectively. The impedance of the insulation 124, 138 and 145 is primarily resistive, and therefore does not vary substantially with frequency.

The following description of the significant benefits and improvements of the relatively high permeability, high resistivity material used in the core 98 of the transformer 90 (FIG. 3) of the present invention, assumes that the same amount of voltage is applied on the secondary winding conductors 100, 139 and 146 of the three transformers 90 (FIG. 3), 130 (FIG. 5) and 141 (FIGS. 6 and 7) represented in FIGS. 8-10 and 11-13, respectively.

One significant benefit is that the insulation 124 on the secondary winding 98 of the transformer 90 of the present invention is required to withstand a small proportion of the high voltage of the electrosurgical output waveform, as may be understood from FIGS. 8 and 11. Under high frequency circumstances, the thin insulation 124 on the secondary winding conductor 100, and the relatively fewer number of secondary windings of the secondary winding conductor 100, and the relatively insignificant inherent core capacitance 156 cause the capacitance effects to be moderate or low. Consequently, the most significant impedance elements are represented by the resistance of the insulation 124 and the inherent core resistance 160, shown by impedance elements 124 and 172 in the divider circuit 168. The relatively high impedance element 172 in the divider circuit 168 absorbs a considerable amount of the high voltage on the secondary winding conductor 100 according to a ratio of the value of the impedance element 124 relative to the value of the sum of the impedance elements 124 and 172. Consequently, the amount of the high voltage on the secondary winding conductor 100 which must be withstood by the impedance element 124 is lowered. As a result, greater immunity from high voltage arcing through the insulation 124 to the core is obtained.

In contrast to the circumstance represented by the low resistivity core 131 of the ferrite power conversion transformer 130 shown in FIGS. 9 and 12, the inherent resistivity 162 of the core 131 is relatively small. That relatively small resistivity 162 is represented by the relatively small impedance element 173 in the divider circuit 170. The relatively small impedance element 173 requires the insulation resistance 138 to withstand a significant amount of the voltage applied on the secondary winding conductor 139. Consequently, relatively thick insulation 138 on the secondary winding conductor 139 is required in order to withstand that relatively high voltage.

In further relation to the characteristics of the plastic-impregnated ferrite particle core transformer 141 (FIGS. 6 and 7) shown in FIGS. 10 and 13, the core 142 exhibits a relatively high inherent resistivity 163, which is shown by the impedance element 174 in the divider circuit 171. The moderate resistance value of the insulation 145 surrounding the secondary winding conductor 146 is shown by the impedance element 145 in the divider circuit 171. The values of the impedance elements 145 and 174 are generally comparable to those values 124 and 172 shown and described in conjunction with the divider circuit 168. Consequently, the high permeability, high resistivity core transformer 90 (FIGS. 2 and 3) of the present invention achieves comparable high voltage withstanding characteristics to those obtained by the typical prior art plastic impregnated ferrite particle transformer 141 (FIGS. 6 and 7). However, the present invention obtains the additional advantages of high permeability that cannot be obtained from the plastic impregnated ferrite particle core 142.

The present invention also obtains advantages in regard to power conversion efficiency and increased bandwidth or spectral energy content of the output electrosurgical waveform, as a result of the relatively low coil-to-coil and coil-to-core parasitic capacitances 148 and 152, as is understood from FIG. 8. The relatively high permeability of the core 96 requires a lesser number of windings 98, and that lesser number of windings reduces the total value of the parasitic capacitances 148 and 152. The inherent capacitance 156 of the high permeability, high resistivity core 96 is also relatively low. The combined effect of these small capacitances 148, 152 and 156 only slightly diminishes the bandwidth or the high frequency spectral energy content of the electrosurgical output waveform conducted by the secondary winding conductor 100. Preserving the high frequency spectral energy content increases the energy conversion efficiency and enhances the electrosurgical effect obtained by the high frequency component of the output of electrosurgical waveform. When the transformer 90 is as a signaling, sensing or isolation transformer (e.g., 24, 26, 28, 30, 32, 34, FIG. 1) the reduced capacitance loading reduces the distortion in the sensed signals generated.

In contrast, the effects of the plastic-impregnated ferrite particle core transformer shown in FIG. 10, the value of the coil-to-coil and coil-to-core parasitic capacitances 151 and 155 are relatively large, due to the relatively large number of windings required on the low permeability core 142. Consequently, at the relatively high frequency of the output of electrosurgical waveform, the high values of these parasitic capacitances 151 and 155 significantly degrade the bandwidth and high frequency spectral energy content of the electrosurgical output waveform, thereby diminishing the energy conversion efficiency and the electrosurgical effects achieved by the high frequency components of the electrosurgical output waveform. Thus, the present invention enhances the high frequency components of the electrosurgical output waveform and increases energy efficiency above that available from the prior art plastic-impregnated ferrite particle transformer, while still obtaining a relatively high permeability which cannot be achieved by the plastic-impregnated ferrite particle transformer.

In general, the values of the coil-to-coil and coil-to-core parasitic capacitances in the transformer of the present invention is generally comparable to that of the ferrite power conversion transformer 130 (FIG. 5) represented in FIG. 9. Thus, the high frequency bandwidth or spectral energy content of the electrosurgical output waveform is generally comparable to that of a prior art power ferrite core transformer. However, the present invention achieves the significant advantages of considerably higher core resistance compared to that available from the prior art power ferrite core transformer, thereby obtaining greatly enhanced immunity to arcing from the secondary winding conductor to the core.

The high permeability, high resistivity core 96, when used as the power output transformer 22 of electrosurgical generator 20 (FIG. 1), is also advantageous in allowing the electrosurgical generator to meet rigorous safety testing standards. While the typical electrosurgical output waveform has a frequency of about 400-600 kHz and several thousand volts, testing of an electrosurgical power output transformer must be performed under the abnormal conditions of a very low test frequency, e.g., 50-60 Hz, and an extremely high test voltage of two times the maximum RF electrosurgical output voltage plus 1,000 volts. Testing at these abnormal conditions of very low frequency and very high voltage is presumed to indicate whether the power output transformer 22 is likely to malfunction and injure the patient or the surgical personnel if an unexpected electrical condition should occur during electrosurgery.

The requirement for the secondary winding of the power output transformer 22 (FIG. 1) to withstand a low frequency, high voltage safety test signal, creates complexities in the construction of the electrosurgical power output transformer. The insulation on the secondary winding must have sufficient thickness and dielectric strength to withstand the exaggerated high voltage of the safety test signal. To assure compliance with safety test requirements, the thickness of the insulation on the secondary winding coils is usually made considerably thicker than is necessary to perform satisfactorily under normal electrosurgical conditions. Of course, increasing the thickness of the insulation on the secondary winding increases the leakage flux, increases the parasitic capacitance and increases the size of the transformer, thereby diminishing the performance of the transformer for the reasons described above.

Because of the safety test requirements, the typical power output transformer for an electrosurgical generator cannot be optimized for electrosurgical performance. However in the case of the present invention, the insulation 124 on the secondary winding 92 withstands the test voltage because of its relatively great dielectric strength per unit of thickness. Use of the high dielectric strength per unit of thickness insulation 124 more closely aligns the safety test response requirements with improved characteristics for normal electrosurgical performance.

The beneficial effects of the present invention under low frequency safety test conditions is also understood by reference to FIGS. 8-10. At relatively low frequencies, the parasitic capacitances 148 and 152, 150 and 154, and 151 and 155 and the inherent core capacitances 156, 158 and 159, are relatively high in value. The relatively high inherent resistivity 160 of the core 96 combines with the high impedance from the capacitances 148, 152 and 156 to cause the impedance element 172 of the divider circuit 168 (FIG. 11) to be relatively high. Again, due to the proportion of the insulation resistance 124 and the impedance element 172, the insulation 124 around the secondary winding is only required to withstand a moderate amount of the relatively high test voltage which is applied to the secondary winding 98.

In contrast, the relatively low resistance of the power conversion ferrite core material 131 of the transformer 130, shown in FIGS. 5 and 9, overshadows the relatively high impedance of its parasitic and inherent core capacitances 150, 154 and 158 at the low test frequency, thereby causing the impedance element 173 of the divider circuit 170 to be relatively low. Again, this effect requires the thickness of the insulation 138 surrounding the high voltage secondary winding conductor 139 to be increased in thickness since the resistance of the insulation 138 must withstand the very high test voltage, causing or accentuating the undesirable characteristics described above.

At the low frequency of the test signal, the plastic-impregnated ferrite particle core 142 (FIGS. 6 and 7) exhibits a relatively high impedance as a result of the relatively high inherent resistivity 159, as shown in FIG. 10. Consequently, the resistivity of the impedance element 174 in the divider circuit 171 (FIG. 11) is relatively high, which permits the insulation 145 to withstand only a moderate amount of the high test voltage applied to the secondary winding conductor 146. In this regard, the low frequency impedance characteristics of the high permeability, high resistivity core material 96 are generally comparable to the very desirable low frequency impedance characteristics of the plastic-impregnated ferrite particle core transformer 130 (FIGS. 6 and 7) while still obtaining the high permeability that can not be achieved by the plastic-impregnated ferrite particle core transformer.

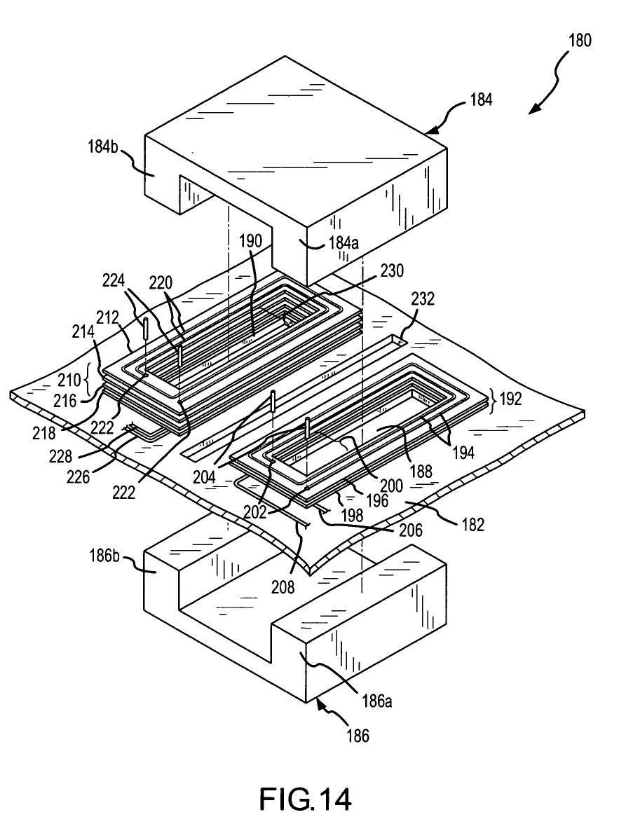

In essence, the use of the high permeability, high resistivity core in a transformer of an electrosurgical generator which conducts or responds to the high frequency electrosurgical output waveform provides substantially all the benefits and substantially none of the detriments of the prior art plastic impregnated ferrite particle core electrosurgical transformer while simultaneously providing substantially all of the benefits and substantially none of the detriments of the prior art power ferrite conversion electrosurgical transformer. In addition, the high permeability high resistivity transformer of the present invention provides increased energy conversion efficiency and increased spectral content of the electrosurgical output waveform which cannot be achieved by either type of prior art in electrosurgical transformer. Further still, the high permeability high resistivity transformer of the present invention allows a reduction in size in an electrosurgical output waveform conducting transformer, as is illustrated in FIG. 14.

Another example of a transformer with a high permeability, high resistivity core that also exhibits increased repeatability in consistent manufacturing tolerances is a printed circuit board transformer 180 illustrated in FIG. 14. The PCB transformer 180 is formed as a part of a principal printed circuit board (PCB) 182 of an electrosurgical generator 20 (FIG. 1). The principal PCB 182 houses and retains many of the functional components 36, 68, 76 and 80 of the electrosurgical generator 20 (FIG. 1), as well as the transformer 180, which may represent one or more of the power output, sense, signaling or isolation transformers 22, 24, 26, 28, 30, 32 or 34 of the electrosurgical generator 20 (FIG. 1).

A core of the transformer 180 is formed by two U-shaped core pieces 184 and 186. The core pieces 184 and 186 are preferably formed from the high permeability and high resistivity core material described above, although there are advantages to the structural integration of the transformer 180 with a PCB 182 apart from the improvements available from the use of the high permeability, high resistivity core material in an electrosurgical transformer. The shape of the core pieces 184 and 186 is comparable to the core pieces 108, 110 or 112, 114 of the representative transformer 90 (FIG. 3). Legs 184a and 184b extend from the core piece 184, and legs 186a and 186b extend from the core piece 186. The core pieces 184 and 186 are oriented so that the ends of legs 184a and 186a contact one another, and the ends of legs 184b and 186b contact one another, when the core pieces 184 and 186 are assembled as the core of the transformer 180. The principal PCB 182 has openings 188 and 190 formed through it. The opening 188 receives the contacting legs 184a and 186a, while the opening 190 receives the contacting legs 184b and 186b. When assembled in this manner, the two U-shaped core pieces 184 and 186 form a closed loop flux flow path through one another, while the PCB 182 extends around and through the closed configuration formed by assembling the two U-shaped core pieces 184 and 186.

The conductors which form a primary winding 192 of the PCB transformer 180 are formed as primary PCB traces 194. Each of the primary PCB traces 194 is formed on smaller primary winding PCBs 196 and 198 using conventional PCB techniques. The primary winding PCB traces 194 encircle a central opening 200 formed in each of the primary winding PCBs 196 and 198. Preferably, the primary PCB trace 194 on each of the PCBs 196 and 198 makes a plurality of completely encircling paths around the central opening 200. The central opening 200 is of approximately the same size as the opening 188 formed in the principal PCB 182. At least one of the legs 184a and 186a of the core pieces 184 and 186 extend through each central opening 200 of the primary winding PCBs 196 and 198. A plurality of the primary winding PCBs 196 and 198 are vertically stacked relative to one another and the principal PCB 182, although two primary winding PCBs 196 and 198 are shown by way of example in FIG. 14. Arranged in this manner, the primary winding PCB traces 194 encircle the legs 184a and 186a of the core pieces 184 and 186.

Each primary winding PCB trace 194 begins and ends at a through hole or via 202 formed in each primary winding PCBs 196 and 198. Vertical interconnects 204 extend through the vias 202 to connect selected ones of the vias 202. The vias 202 are formed at slightly different locations on adjacent pairs of the PCBs 196 and 198, so that the vertical interconnects 204 can connect the PCB traces 194 in series with one another. Similarly, traces 206 and 208 are formed in the principal PCB 182, and the traces 206 and 208 end in vias (not shown). Vertical interconnects 204 extend through the vias in the traces 206 and 208 and the vias 202 at the beginning and ending ones of the PCB traces 194 which begin and end the series connection of those PCB traces 194 of the primary winding 192. A primary signal is supplied to the traces 206 and 208, and that primary signal flows through the vertical interconnects 204, through the vias 202 and through the series-connected PCB traces 194 on the primary winding PCBs 196 and 198, thus completing a current flow path through the primary winding 192.

The secondary winding 210 of the transformer 180 has a similar construction to the primary winding 192, except that more secondary winding PCBs 212, 214, 216 and 218 are employed (four are shown). Each of the PCBs 212, 214, 216 and 218 is formed with its own secondary winding PCB trace 220. Through holes or vias 222 are formed at the ends of the PCB traces 220 on the secondary winding PCBs 212, 214, 216 and 218, and vertical interconnects 224 extend through those vias 222 to connect the secondary winding PCB traces 220 in series with one another among the secondary winding PCBs 212, 214, 216 and 218. Traces 226 and 228 are formed on the principal PCB 182, and the traces 226 and 228 include vias (not shown) which are connected by vertical interconnects 224 to the vias 222 which define the beginning and end of the series connected secondary winding traces 220 of the PCBs 212, 214, 216 and 218. The secondary winding traces 226 and 228 are also preferably formed in multiple complete encircling paths surrounding a central opening 230 on each of the PCBs 212, 214, 216 and 218. The number of completely encircling traces 200 on each PCB 212, 214, 216 and 218 may be greater than the number of completely encircling traces on the primary winding PCBs 196 and 198. At least one of the legs 184b and 186b of the core pieces 184 and 186 extend through the central opening 230 in each of the PCBs 212, 214, 216 and 218. As a result, the secondary winding PCB traces 220 also encircle the high permeability, high resistivity core pieces 184 and 186. A secondary signal is conducted by the traces 226 and 228, and that is secondary signal flows through the vertical interconnects 224, through the vias 222 and through the series-connected secondary PCB traces 220 on the secondary winding PCBs 212, 214, 216 and 218, thus completing a signal flow path through the secondary winding 210.

The primary winding PCBs 196 and 198 and the secondary winding PCBs 212, 214, 216 and 218 are held relative to one another and to the principal PCB 182 by the vertical interconnects 204 and 224 and by an adhesive or a support structure (neither shown). The core pieces 184 and 186 are held together with the ends of the legs 184a, 186a and 184b, 186b contacting or adjacent to one another by a conventional retention device (not shown) or by an adhesive. A central slot 232 extends through the principal PCB 182 between the central openings 200 and 230 to provide an air gap with increased dielectric strength for separating the primary and secondary windings 192 and 210 from one another.

The number of windings (traces 194 and 220) of the primary and secondary windings 192 and 210 are established by the number of encirclements of each trace on each primary and secondary PCB, and by the number of primary and secondary PCBs which are stacked and interconnected with one another in the manner described. The traces 192 and 210 can be formed on both sides of each primary and secondary PCB to the reduce the number of primary and secondary PCBs and/or to increase the number of windings. Under such circumstances, a layer of insulation (not shown) or an air gap (which is shown in FIG. 14) is provided between the vertically spaced primary and/or secondary PCBs to insulate the adjacent traces and the PCBs from one another.

Because of the close manufacturing tolerances attainable by using printed circuit board techniques, the windings can be spaced very closely adjacent to the legs of the core pieces and spaced closely adjacent to one another by vertically stacking the primary and secondary PCBs. The PCB transformer 180 can be integrated in construction with the principal PCB board 182, which will normally achieve cost reductions compared to using an alternative non-PCB transformer in an electrosurgical generator. A high level of manufacturing repeatability for both the PCB transformer 180 and other forms of the transformer 90 are achieved because of reduced variations of physical placement and orientation of their components. The precise nature of the placement of the primary and secondary windings, along with the use of the high permeability, high resistivity core pieces 184 and 186, significantly enhance the practical applications for a transformer which conducts the electrosurgical output waveform in an electrosurgical generator.

The high permeability, high resistivity core, combined with the relatively thin, high dielectric strength insulation for the winding conductors results in a significantly improved transformer for an electrosurgical generator and a significantly improved electrosurgical generator. Greater energy conversion efficiency results from the higher permeability of the core compared to lower permeability cores typically used in electrosurgical output power, sensing, signaling and isolation transformers which conduct and respond to the high frequency, high voltage electrosurgical output waveform. The higher permeability permits less leakage flux to escape from the core without interacting with the windings. The thinner insulation positions the windings closer to the core and thereby limits the space for the leakage flux to escape. The higher permeability reduces the number of coils or turns of the windings around the core, thereby reducing the size of the transformer. The greater amount of mutual flux interacting with the windings increases the voltage of the induced signal without requiring additional coils of the winding. The reduced number of winding coils diminishes the energy loss caused by current flowing through longer windings. The reduced number of coils of the winding also reduces the parasitic coil-to-coil and coil-to-core capacitances created by the winding, because fewer coils of the winding create these undesired parasitic capacitances. The high frequency energy content of the electrosurgical output waveform is enhanced because less high frequency energy is consumed in charging the smaller parasitic capacitances. Less leakage current improves electrosurgical performance and diminishes the risk to surgical personnel. A greater bandwidth of energy is available for electrosurgery. The relatively high resistivity of the core is capable of supporting a higher voltage on the high voltage secondary winding without arcing or discharging to the core through the insulation on the high-voltage winding conductor.

The level of enhanced performance available from the high permeability, high resistivity transformer offers the possibility of using a single power output transformer in electrosurgical generator to perform both cutting and coagulation. Some prior art electrosurgical generators use one type of power output transformer for cutting and a different type of power output transformer for coagulation. Tissue cutting is viewed as more akin to power conversion, and for that reason the power output transformer for cutting may utilize a high permeability, low resistivity power ferrite core power output transformer. The low resistivity would generally not have a significant effect during cutting because the electrical load imposed by cutting inherently diminishes the output voltage. The degraded high frequency response characteristics do not particularly adversely influence cutting. On the other hand, achieving the best coagulation is usually dependent on preserving the high frequency energy spectral content of the electrosurgical output waveform. For this reason, a plastic-encapsulated ferrite particle transformer is typically used as the power output transformer for coagulation. The compromise available from the high permeability, high resistivity transformer of the present invention offers enough significant advantages for both cutting and coagulation that a single power output transformer 22 (FIG. 1) with a high permeability, high resistivity core 96 (FIG. 3) may replace both of the prior art transformers in a single electrosurgical generator, thereby reducing the cost and size of the electrosurgical generator.

Governmental standards which regulate the safety of electrosurgical generators require narrow tolerances on the output voltages and powers delivered from the power output transformer. Practical variations in parasitic winding capacitances and/or energy storage characteristics of the windings and the core makes achieving these tolerances very difficult, particularly when a large number of winding turns or coils are required. The same problem of close tolerances also applies to sensing, signaling and isolation transformers which conduct or respond to the electrosurgical output waveform. The reduced number of windings of more thinly insulated wire in combination with the high permeability, high resistivity core, make these tolerances easier to achieve by providing the opportunity to manufacture transformers repeatable and controllable precision. The manufacturability of a transformer using the high permeability, high resistivity core is therefore greatly enhanced.

Many other advantages, benefits and improvements will be apparent upon fully understanding the ramifications of the present invention. Presently preferred embodiments of the invention and many of its improvements have been described with a degree of particularity. This description is a preferred example of implementing the invention, and is not necessarily intended to limit the scope of the invention. The scope of the invention is defined by the following claims.

The invention claimed:

1. In an electrosurgical generator which delivers a high frequency, high voltage electrosurgical output waveform for use in an electrosurgical procedure performed on a patient, a transformer comprising a winding which conducts the electrosurgical output waveform and a core which the winding encircles, and an improvement comprising:
material forming the core which has a permeability in the range of 500-2000 and a resistivity in the range of 90,000-1,000,000 ohm centimeters.

2. An improved electrosurgical generator as defined in claim 1, wherein:
the material forming the core has permeability in the range of approximately 800-2000 and resistivity in the range of 100,000-1,000,000 ohm centimeters.

3. An improved electrosurgical generator as defined in claim 1, wherein:
the material forming the core has permeability in the range of 800-2000.

4. An improved electrosurgical generator as defined in claim 1, wherein:
the material forming the core has resistivity in the range of 100,000-1,000,000 ohm centimeters.

5. An improved electrosurgical generator as defined in claim 1, wherein the winding is a secondary winding which is formed by a secondary electrical conductor which is covered with electrical insulation having a dielectric strength of at least 800 VAC per 0.001 inch thickness of the insulation.

6. An improved electrosurgical generator as defined in claim 1, wherein the secondary winding is formed by a secondary electrical conductor which is covered with electrical insulation having a dielectric strength in the range of 800-2000 VAC per 0.001 inch thickness of the insulation.

7. An improved electrosurgical generator as defined in claim 1, wherein the secondary winding is formed by a secondary electrical conductor which is covered with electrical insulation having a substantially uniform thickness of approximately 0.006 inch.

8. An improved electrosurgical generator as defined in claim 1, wherein the secondary winding is formed by a secondary electrical conductor which is covered with electrical insulation formed from a fluoropolymer.

9. An improved electrosurgical generator as defined in claim 1, wherein the secondary winding is formed by a secondary electrical conductor which is covered with multiple uniform thickness layers of electrical insulation.

10. An improved electrosurgical generator as defined in claim 9, wherein:
each of the layers has a thickness of about 0.002 inches.

11. An improved electrosurgical generator as defined in claim 9, wherein:
each of the layers is formed of a fluoropolymer.

12. An improved electrosurgical generator as defined in claim 1, wherein:
the transformer is a power output transformer, the winding is a secondary winding of the power output transformer, and the secondary winding produces the electrosurgical output waveform.

13. An improved electrosurgical generator as defined in claim 12, wherein:
a single one power output transformer produces the electrosurgical output waveform which is suitable for both electrosurgical cutting and electrosurgical coagulation.

14. An improved electrosurgical generator as defined in claim 1, wherein:
the transformer is a sensing transformer which senses one of the voltage or current of the electrosurgical output waveform.

15. An improved electrosurgical generator as defined in claim 1, wherein:
the transformer is one of a signaling, sensing or isolation transformer, the winding is a secondary winding of the signaling, sensing or isolation transformer, and the signaling, sensing or isolation transformer further includes a primary winding which supplies a signal derived from the electrosurgical output waveform.

16. An improved electrosurgical generator as defined in claim 15 in which a return electrode is connected to the patient, wherein:
the primary winding supplies a monitoring signal conducted by the return electrode which represents a degree of contact of the return electrode with the patient.

17. An improved electrosurgical generator as defined in claim 15 in which the electrosurgical output waveform is delivered from an active electrode retained on a handpiece that has a switch for selecting a mode of electrosurgical operation, wherein:
the primary winding supplies a mode signal which is conducted from the electrosurgical output waveform by the switch.

18. An improved electrosurgical generator as defined in claim 1 in which a principal printed circuit board (PCB) houses and retains electrical components of the electrosurgical generator, and wherein:
the core of the transformer extends through an opening in the principal PCB;
the winding comprises a plurality of PCB traces which encircle the core of the transformer; and
the plurality of PCB traces which form the winding are supported by the principal PCB.

19. An improved electrosurgical generator as defined in claim 18, further comprising:
an additional PCB in addition to the principal PCB, the additional PCB having an opening formed therein which encircles a portion of the core of the transformer; and wherein:
the plurality of PCB traces which form the winding are formed on the additional PCB surrounding the opening in the additional PCB and encircle a portion of the core of the transformer; and
the additional PCB is retained by the principal PCB.

20. An improved electrosurgical generator as defined in claim 19, in which the principal PCB includes traces, and wherein the plurality of PCB traces on the additional PCB are connected to the principal PCB traces.

21. In an electrosurgical generator which delivers a high frequency, high voltage electrosurgical output waveform for use in an electrosurgical procedure performed on a patient, a transformer comprising a core around which primary and secondary windings are wound, the secondary winding formed by a secondary electrical conductor which is covered with electrical insulation, the secondary electrical conductor conducting the electrosurgical output waveform, and an improvement wherein:
the electrical insulation covering the secondary electrical conductor has a substantially uniform thickness and has multiple layers.

22. An improved electrosurgical generator as defined in claim 21, wherein:
each of the layers has a uniform thickness and the thickness of each layer is about 0.002 inches.

23. An improved electrosurgical generator as defined in claim 21, wherein:
each of the layers is formed from a fluoropolymer.

24. An improved electrosurgical generator as defined in claim 21, wherein:
each of the layers as a dielectric strength of at least 800 VAC per 0.001 inch thickness.

25. A method of increasing the high frequency energy content of a high frequency, high voltage electrosurgical output waveform delivered from an electrosurgical generator to a patient-referenced circuit to perform electrosurgery on a patient, while simultaneously reducing leakage current from the electrosurgical output waveform and enhancing the resistance to arcing and glow discharge of the high-voltage electrosurgical output waveform, comprising:
utilizing a transformer with a core having a permeability in the range of 500-2000 and a resistivity in the range of 90,000-1,000,000 ohm centimeters; and
conducting the electrosurgical output waveform through a secondary winding of the transformer which encircles the core.

26. A method as defined in claim 25, further comprising:
insulating an electrical conductor which forms the secondary winding with electrical insulation having a dielectric strength in the range of 800-2000 VAC per 0.001 inch of thickness of insulation.

27. A method as defined in claim 25, wherein the transformer also includes a primary winding encircling the core, and the method further comprises:
inducing the electrosurgical output waveform from the secondary winding by applying a signal to the primary winding.

28. A method as defined in claim 25, wherein the transformer also includes a primary winding encircling the core, and the method further comprises:
sensing a signal at the primary winding which has been superimposed on the electrosurgical output waveform.

29. A method of increasing resistance to arcing and glow discharging through electrical insulation surrounding a secondary winding conductor which encircles a core of a power output transformer of an electrosurgical generator, comprising:
insulating an electrical conductor which forms the secondary winding with multiple layers of electrical insulation with each layer having a dielectric strength in the range of 800-2000 VAC per 0.001 inch of thickness.

30. A method as defined in claim 29, further comprising:
utilizing material for the core which has a permeability in the range of 500-2000 and a resistivity in the range of 90,000-1,000,000 ohm centimeters.

31. A method as defined in claim 29, which also enhances the capability of withstanding a high voltage safety test in which there is applied to the secondary winding a test signal having a voltage of at least two times a highest expected maximum voltage of the electrosurgical output waveform and having a frequency of approximately 50-60 hertz.
